(19) United States
(12) Patent Application Publication
(10) Pub. No.: US 2004/0043055 A1
(43) Pub. Date: Mar. 4, 2004

(54) Method of reducing body weight and/or body fat with L-glutamine compounds

(75) Inventor: Takeo Ueda, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 10/229,012

(22) Filed: Aug. 28, 2002

(51) Int. Cl. A61K 31/198; A61K 38/00

(52) U.S. Cl. 424/439; 514/2; 514/564

(57) ABSTRACT

A method and compositions for reducing body weight, body fat, or both.

### BACKGROUND OF THE INVENTION

1. Field of the Invention

The present invention relates to methods and compositions for reducing body weight, body fat, or both.

2. Description of the Background

There remains a need for compositions and methods for reducing body weight, body fat, or both. Compositions containing L-glutamine have been used for this purpose. For example, it has been reported that addition of 3.6% of L-glutamine to the basal diet of mice inhibited body weight gain (Opara et al., J. Nutr. 126:273 (1996)). In addition, WO 95/11019 describes a beverage composition which contains L-glutamine in an amount from 1 to 4 g per 473 milliliters of the beverage which is reported to enhance physical performance and reduce body fat by inducing a growth hormone response.

However, there remains a need for improved compositions for reducing body weight and/or body fat.

### SUMMARY OF THE INVENTION

It is an object of the present invention to provide a method for reducing body weight, body fat, or both.

It is another object of the invention to provide compositions for reducing body weight, body fat, or both.

The present invention is based on the discovery that ingesting an L-glutamine compound at a daily level higher than hitherto reported and physically exercising provides enhanced reduction in body weight, body fat, or both.

Thus, the objects of the invention, and others, may be accomplished with a method of reducing body weight and/or body fat, comprising:

administering to a subject at least 10 g of an ingestible L-glutamine compound per day; and

subjecting the subject to physical exercise.

The objects of the invention may also be accomplished with a method of reducing body weight and/or body fat, comprising:

consuming at least 10 g of an ingestible L-glutamine compound per day; and

physically exercising.

The objects of the invention may also be accomplished with a liquid beverage product, comprising at least 5 g of a L-glutamine compound per 473 mL of the beverage product.

### BRIEF DESCRIPTION OF THE FIGURES

A more complete appreciation of the invention and many of the attendant advantages thereof will be readily obtained as the same becomes better understood by reference to the following detailed description considered together with the following Figures:

FIG. 1: relative body weight to the LF group in week 5 in the experiment described in Example 1 herein;

FIG. 2: relative body weight to the LF group in week 10 in the experiment described in Example 1 herein;

FIG. 3: relative body weight to the LF group in week 16 in the experiment described in Example 1 herein;

FIG. 4: relative fasting plasma insulin concentration to the LF group in week 16 in the experiment described in Example 1 herein;

FIG. 5: relative liver glycogen content after 24 h of starvation in week 16 of the experiment described in Example 1 herein;

FIG. 6: relative body weight to the LF group in week 6 of the experiment described in Example 2 herein;

FIG. 7: relative weight of epididymal fat to the LF group in week 6 of the experiment described in Example 2 herein; and

FIG. 8: putative effects of the combination of glutamine and exercise on body weight and body fat reduction.

### DETAILED DESCRIPTION OF THE INVENTION

The present invention provides a method of reducing body weight and/or body fat, by administering at least 10 g of an ingestible L-glutamine compound per day to a subject, and having the subject perform physical exercise. Thus, the method of the invention can produce a reduction in body weight, a reduction in body fat, or both.

As used herein, the term “ingestible” means that the material can be safely consumed by a subject particularly a human subject. In particular, as will be described in greater detail below, the L-glutamine compound is preferably consumed in the form of a food or beverage product.

An ingestible L-glutamine compound is consumed by the subject. As used herein, the term “L-glutamine compound” refers to a L-glutamine itself (or a salt thereof) or a compound which functions as a physiologically acceptable source of L-glutamine. Thus, the ingestible L-glutamine compound may L-glutamine, or it may be a peptide containing L-glutamine, or a physiologically acceptable derivative of L-glutamine, or a salt thereof. A preferred L-glutamine compound is L-glutamine.

Peptides containing L-glutamine as a constituent amino acid are well known. Peptides rich in L-glutamine are particularly preferred. Where the L-glutamine is supplied in the form of peptides rich in L-glutamine, the L-glutamine content of the peptide should be sufficiently high to secure the beneficial effect of L-glutamine. The peptides to be employed in the method of the invention therefore preferably contain at least 20% by weight, preferably more than 24% by weight of L-glutamine. Suitable peptide sources for L-glutamine include short chain peptides such as dipeptides, e.g. L-alanyl-L-glutamine (which contains ca. 63% by weight of L-glutamine), tripeptides, tetrapeptides, pentapeptides, hexapeptides, longer chain peptides or peptide mixtures, e.g. in the form of isolated proteins rich in L-glutamine, hydrolysates of L-glutamine rich proteins or appropriate fraction of such proteins or hydrolysates. Typical examples of L-glutamine rich protein sources are for example wheat (which is a source for gliadin, a protein containing 35 to 43% of glutamine) and beans (of which the seed storage protein contains ca. 24% of glutamine).

In a preferred embodiment the peptides rich in L-glutamine are substantially in short chain form and contain from 2 to 10, preferably from 2 to 8, more preferably from 2 to 6, particularly from 2 to 4 amino acids.

Other physiologically acceptable L-glutamine derivatives are known in the art. They include L-glutamine salts, N-acyl derivatives of L-glutamine including N-alkanoyl L-glutamine compounds such as N-acetyl L-glutamine. The N-acylation of L-glutamine stabilises, and therefore prevents decomposition of L-glutamine to L-pyroglutamate. Physiologically acceptable derivatives of L-glutamine that may be used in this invention include:

L-glutamine alkyl ester (alkyl ester: C=1-22)

L-glutamine alkenyl ester (alkenyl ester: C=2-22)

L-glutamine aralkyl ester (aralkyl ester: C=7-22)

L-glutamine amide

N-alkyl L-glutamine (alkyl residue: C=1-22)

N-alkenyl L-glutamine (alkenyl residue: C=2-22)

N-aralkyl L-glutamine (aralkyl residue: C=7-22)

N-alkyl L-glutamine alkyl or alkenyl or aralkyl ester

(alkyl residue: C=1-22, alkenyl residue: C=2-22, aralkyl residue: C=7-22)

N-alkenyl L-glutamine alkyl or alkenyl or aralkyl ester

(alkyl residue: C=1-22, alkenyl residue: C=2-22, aralkyl residue: C=7-22)

N-aralkyl L-glutamine alkyl or alkenyl or aralkyl ester

(alkyl residue: C=1-22, alkenyl residue: C=2-22, aralkyl residue: C=7-22)

N-alkyl L-glutamine amide (alkyl residue: C=1-22)

N-alkenyl L-glutamine amide (alkenyl residue: C=2-22)

N-aralkyl L-glutamine amide (aralkyl residue: C=7-22)

N-acyl L-glutamine alkyl or alkenyl or aralkyl ester

(acyl residue: C=1-22, alkyl residue: C=1-22, alkenyl residue: C=2-22, aralkyl residue: C=7-22)

N-acyl L-glutamine amide (acyl residue: C=1-22)

The range for the carbon numbers listed above include all specific values and subranges therebetween, such as 2, 4, 6, 8, 10, 12, and 18 carbon atoms.

The amount of the ingestible L-glutamine compound is consumed by the subject is at least 10 g per day. Preferably, 10 to 50 g of the ingestible L-glutamine compound is administered to the subject per day. Even more preferably, 10 to 30 g of the ingestible L-glutamine compound is administered to the subject per day. Still more preferably, 10 to 20 g of the ingestible L-glutamine compound is administered to the subject per day. These ranges include all specific values and subranges therebetween, such as 15, 25, 35, 40, and 45 g of the L-glutamine compound per day.

The ingestible L-glutamine compound may be administered to the subject in a wide variety of forms. Thus, the L-glutamine compound is administered in the form of an ingestible composition. In one embodiment, the L-glutamine compound is administered in the form of a tablet, capsule, or concentrate. In another embodiment, the L-glutamine compound is administered in the form of a liquid drink product. In yet another embodiment, the L-glutamine compound is administered in the form of a food. When the subject is an animal, the ingestible composition may be a pet food or a feed additive. Suitable drink products include carbonated and non-carbonated beverage products, liquid concentrates, ready-to-drink liquid products, and powdered beverage products. Suitable food products include candy bars, energy bars, nutrition bars, cookies, crackers, lozenges, chocolates, drops, jellies, sweets and the like. The base composition of such products are well-known in the art.

The liquid product of the present invention contains at least 5 g of L-glutamine per 473 milliliters (16 fluid ounces). Preferably, such a product contains at least 8 g of L-glutamine per 473 milliliters up to the solubility limit (22.7 g, 12.3, and 8.3 g per 473 milliliters at 30, 18, and 0° C., respectively.

The ingestible composition may contain a wide variety of additional additives which are customarily incorporated into food and beverage products. Such additives include vitamins (vitamin A, vitamin C, vitamin E, vitamin B₂, vitamin B₆, panthothenic acid, nicotinic acid, etc.), sweetening agents (aspartame, sugar, etc), organic acids (citric acid, malic acid, fumaric acid, malonic acid, succinic acid, tartaric acid, lactic acid, etc), coloring agents, flavoring agents (vanillin, linalool, natural perfumes, etc), anti-wetting agents, fibers, electrolytes, minerals, nutrients, antioxidants, preservatives, aromas, humectants, natural plant extracts (tea extracts, coffee extracts, cocoa extracts), fruit extracts (such as orange, grape, apple, peach, pineapple, pear, plum, cherry, papaya, tomato, melon, strawberry, raspberry, etc).

As noted above, the composition of these products is well-known, and one skilled in the art will readily appreciate the types of additives that are customarily used in such products.

The subject may be a human or an animal. Suitable animals include horses and dogs. Humans are preferred subjects.

The ingestible L-glutamine compound may be administered prior to or after exercise. It is contemplated that the methods of the present invention may be used for weeks, months, or years. Thus, the subject will consume the L-glutamine compound at the indicated daily dose and continue to exercise during this time. Accordingly, the time period of the inventive method may be at least two weeks, at least one month, at least 3 months, at least 6 months, at least 1 year, at least 2 years, at least 3 years, at least 5 years, or at least 7 years. It is within the scope of the present invention that the inventive method will be used throughout the life span. Thus, a permanent regimen of consumption of the L-glutamine compound and physical exercise.

In a preferred embodiment of the invention, the exercising is conducted at at least the 40% VO₂max of the subject. More preferably, the exercising is conducted at 40% to 85% VO₂max. Still more preferably, the exercising is conducted at 40% to 70% VO₂max. Even more preferably, the exercising is conducted at 50% to 70% VO₂max.

In another preferred embodiment, the exercising is conducted for at least 20 minutes. The physical activity is preferably aerobic. Suitable activities include walking, running/jogging, swimming, bicycling, and boating.

As described above, edible compositions comprising L-glutamine, in free amino acid form, or in peptide form, or in the form of another physiologically acceptable L-glutamine derivative reduce both body weight and body fat deposit, and prevent obesity synergistically in the combination with physiological activities such as sports, walking, jogging, aerobic movements and the like. Without being limited to any particular theory, L-glutamine is believed to reduce insulin secretion from the pancreas as a result of enhanced glucose utilization in the muscles, which results in a reduction in lipogenesis. Exercise, an energy-consuming event, metabolizes glycogen and, thereafter, free fatty acid. Reduced insulin secretions resulting from L-glutamine administration also accelerates glycogenolysis and thereafter stimulates lipolysis induced by exercise. Therefore, the combination of L-glutamine consumption and exercise can exert synergistic effects on reduction in both body weight and body fat depot, and on preventing obesity.

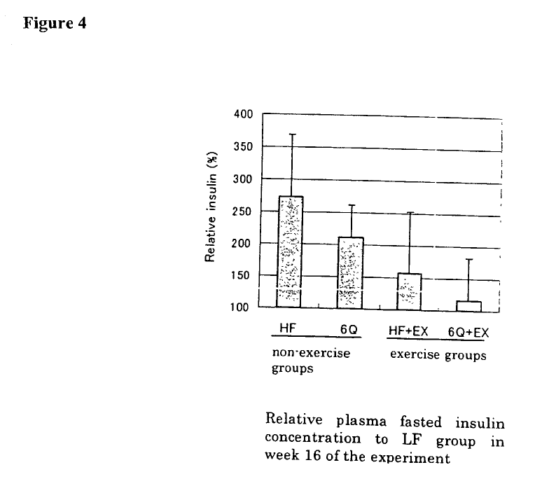

Referring to the Examples described hereinafter, plasma concentrations of insulin, only one anabolic hormone animals possess, were considerably high in HF treatment compared with LF treatment. Either L-glutamine administration or exercise reduced plasma insulin concentrations by high fat feedings. The value in the group of the combination of L-glutamine administration and exercise was the lowest and was close to that of LF group (FIG. 4).

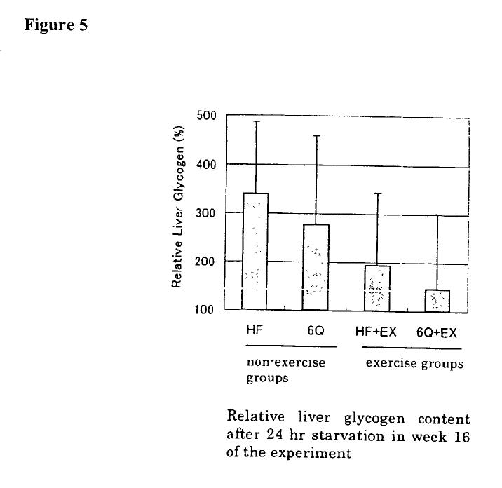

The results of glycogen contents in the liver after starvation were the same trends as plasma insulin concentrations (FIG. 5). The HF group had the highest remaining glycogen content and L-glutamine plus exercise had the lowest.

These results suggested that changes of the metabolism by high fat feedings were mostly prevented by the combination of L-glutamine administration and exercise and that the status of the body remained catabolic as compared to mice fed normal diet.

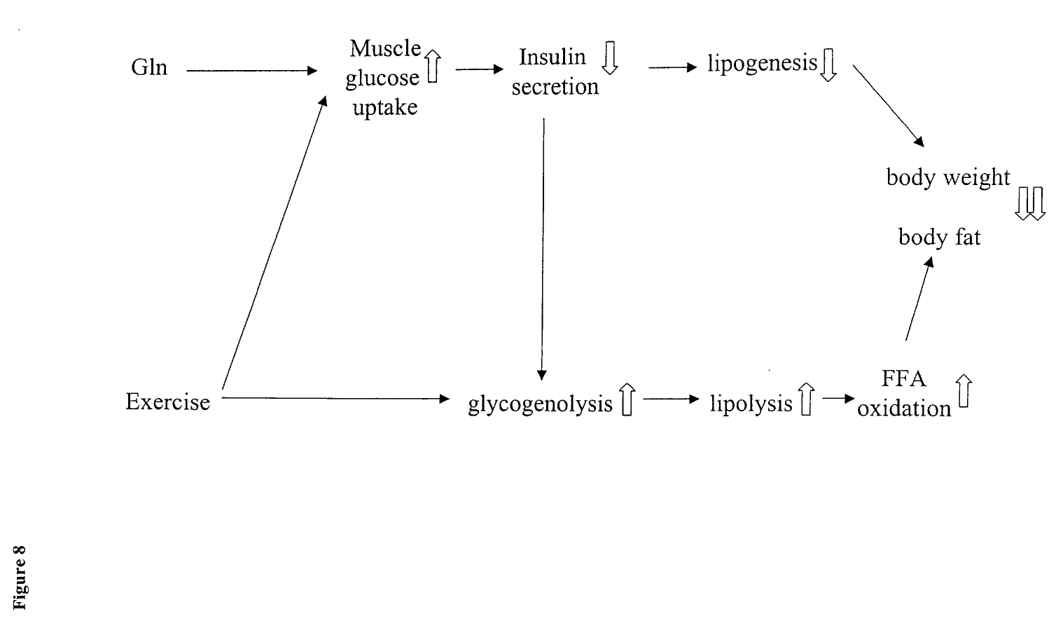

Again, without being limited to any particular theory, a hypothesis for the mechanism of the combination of L-glutamine administration and exercise on body weight and body fat reduction is shown in FIG. 8.

Both L-glutamine and exercise enhance glucose utilization in muscle and, as a result, reduce insulin secretion from pancreas (as shown in FIG. 4), which results in reduction of lipogenesis.

Exercise, an energy-consuming event, metabolizes glycogen (glycogenolysis; as shown in FIG. 5) which is also enhanced by lower plasma insulin levels by L-glutamine feeding. After the glycogen store disappears, free fatty acids (FFA) are mobilized (lipolysis) and FFA oxidation is induced.

Therefore, the combination of L-glutamine administration and exercise exerts synergistic effects on reductions of body weight and body fat depots and on prevention body weight and body fat gain.

To apply the data obtained from the Examples described herein to humans, utilization of the ratios of L-glutamine to protein content at 20.3% in the diets in the Examples appears to be the most appropriate. Based upon the current Recommended Dietary Allowance (RDA) for protein for humans, 0.8 g protein/kg/day is referred as the safe and adequate intake for virtually all healthy men and women aged 19 years and older. Therefore, when daily L-glutamine amounts to be administered for humans were calculated by using this number, 0.14 (=0.8×3.6/20.3) and 0.24 (=0.8×6.0/20.3) g/kg/day corresponded to 3.6% and 6.0%, respectively. Assuming of 60 kg of body weight, 8.5 g of L-glutamine is to be taken in the case of the 3.6% content reported by Opara et al. which is the same range from 5 to 10 g of L-glutamine per day usually recommended to enhance muscle performance during exercise. In case of the dosage of 6.0% at which the synergistic effects were obtained in the Example 1, the amount was determined as 14 g/day, which is above the range normally recommended for athletes. Therefore, dosage for body weight and body at regulation is suitable at 10-50 g/day, preferably at 10-30 g/day, most preferably at 10-20 g/day in human.

In addition, WO 95/11019 describes that nutritional compositions comprising L-glutamine reduce body fat without exercise through the induction of growth hormone response at a relatively low dose of L-glutamine at 1.0 to 4.0 g. It is noted that higher doses than the range may negate the beneficial effect of the glutamine. Since effective dosage in the present invention is much higher than that described above, it is clear that the mechanism as well as the optimal dosage were totally different from those described in WO 95/11019.

### EXAMPLES

Having generally described this invention, a further understanding can be obtained by reference to certain specific examples which are provided herein for purposes of illustration only and are not intended to be limiting unless otherwise specified.

### Example 1

Synergistic Effects of L-glutamine Administration Combined with Exercise

Fifty male C57BL/6J mice, 9 weeks old, were used herein. The animals were assigned into one of five treatments, 1) normal fat diet (LF), 2) high fat diet (HF), 3) high fat diet containing 6.0% of L-glutamine (6Q), 4) high fat diet plus exercise (HF+EX), and 5) high fat diet containing 6.0% of L-glutamine plus exercise (6Q+EX). The experiment was conducted for 16 weeks. The mice exercised on a treadmill at moderate intensity (at 4.0 m/min) for 30 minutes a day, 5 days a week. It is known that oxygen consumption (VO₂) strongly correlates with exercise intensity. However, VO₂ reaches the plateau at a certain point as exercise intensity increases, which is defined as maximum oxygen consumption (VO₂max). Percentage of maximum oxygen consumption (% VO₂max) is one of the common ways to indicate the exercise intensity. According to Schefer V. and Talan M. I. (Exp. Gerontol. 31:387 (1996)), a running speed of 3-4 m/min which was the same intensity of exercise used herein produced a VO₂ equivalent to 54-58% of the VO₂max in adult C57BL/6J mice. In 16 weeks of the experiment, plasma and liver samples were obtained after overnight starvation.

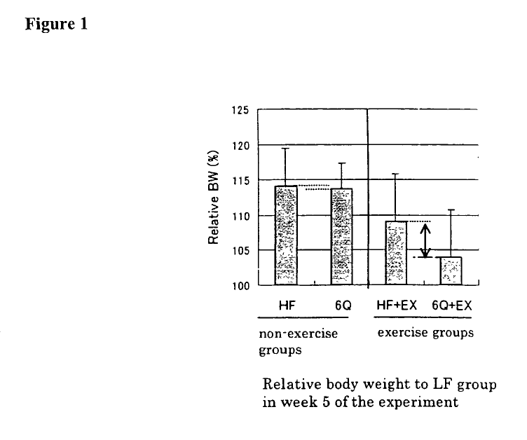

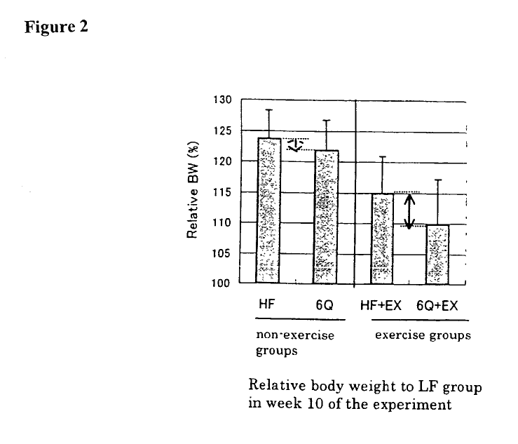

Relative body weight to the LF group in week 5, 10, and 16 are shown in FIGS. 1, 2, and 3, respectively. In week 5, L-glutamine administration did not reduce the rate of body weight gain in non-exercise groups (FIG. 1). In exercise groups, however, body weight gain by high fat feeding prevented considerably in L-glutamine fed mice (FIG. 1). This synergistic effect lasted for 10 weeks of the experiment (FIG. 2).

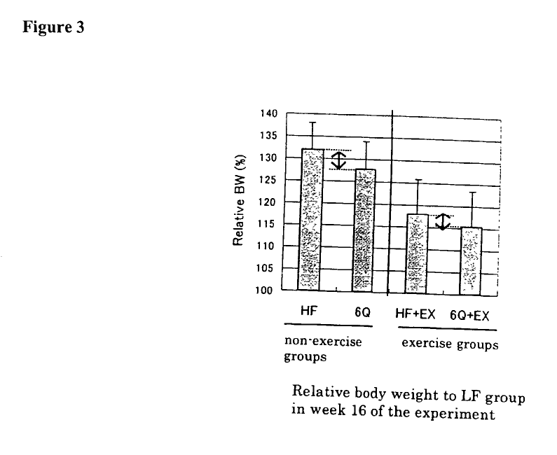

The combined effects disappeared in week 16, although lower body weight was still observed in 6Q+EX group compared to HF+EX group (FIG. 3). The reason might be that mice were becoming metabolically adapted to L-glutamine feeding so that the L-glutamine was metabolized more rapidly in week 16 than in the early stage of the experiment. As the life span of mice is rather short, approximately 2 years, the feeding period of L-glutamine for 4 months was considerably long relative to their life span. Applying to a human, assuming a life span of 70 years, 10 weeks and 4 months for mice correspond to 7 and 12 years for a human, respectively. Therefore, the synergetic effects of L-glutamine combined with exercise may possibly be exerted for at least 7 years in human without diminution by this metabolic adaptation.

As described above, it is known that oxygen consumption strongly correlates with exercise intensity. In addition, the relationship between the log-log plot of maximum oxygen consumption (% VO₂max) and body mass for 37 species of mammals is highly correlated (Jones J. H. and Lindstet S. L. Annu. Rev. Physiol. 55:547 (1993), incorporated herein by reference). In addition, the resting oxygen consumption at 28.2% VO₂max in adult C57BL/6J mice (Schefer V. and Talan M. I. Exp. Gerontol. 31:387 (1996)) is close to oxygen consumption in normal life in human at 30 to 40% VO₂max (Iio Y. et al., Nutrition and Exercise and Rest. pp. 107 (1999) Koseikan, Tokyo, Japan). Therefore, it is considered that % VO₂max would be the most appropriate way to refer the exercise intensity of mice to that of human.

It is reported that more than 85% VO₂max promotes carbohydrate oxidation and does not increase lipid utilization (Coyle E. F. Am. J. Clin. Nutr. 61 (suppl): 968S (1995)). Thus, to increase fat utilization efficiently, exercise intensity is suitable at 40-85% VO₂a max, preferably at 40-70% VO₂max, and most preferably at 50-70% VO₂max.

Based upon the recommendations of American College of Sports Medicine, duration of the exercise is considered to be proper for more than 20 min.

### Example 2

Reduction of Body Weight, Epididymal Fat, and Abdominal Fats

Forty of male C57BL/6J mice, age 5 weeks, were used herein. The animals were assigned into one of four treatments, 1) normal fat diet (LF), 2) high fat diet (HF), 3) high fat diet containing 3.6% of L-glutamine (HF+4Q), 4) high fat diet containing 6.0% of L-glutamine (HF+6Q). After 6 weeks of the diet feedings, body weight and epididymal fat weight were measured.

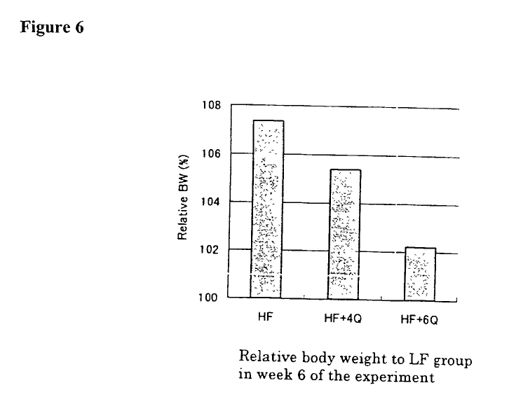

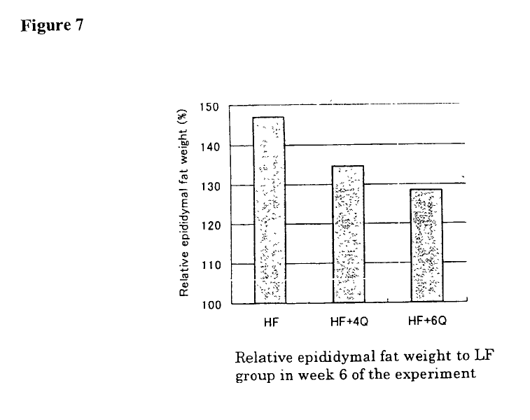

The rates of body weight gain were reduced by L-glutamine administrations in a dose-dependent manner (FIG. 6). In addition, weights of epididymal fat and abdominal fats also decreased in a dose-dependent manner (FIG. 7).

Obviously, numerous modifications and variations of the present invention are possible in light of the above teachings. It is therefore to be understood that within the scope of the appended claims, the invention may be practiced otherwise than as specifically described herein.

1. A method of reducing body weight and/or body fat, comprising:
administering to a subject at least 10 g of an ingestible L-glutamine compound per day; and
subjecting the subject to physical exercise.

2. The method of claim 1, wherein the ingestible L-glutamine compound is L-glutamine, a peptide containing L-glutamine, or a physiologically acceptable derivative of L-glutamine.

3. The method of claim 1, wherein the ingestible L-glutamine compound is L-glutamine.

4. The method of claim 1, wherein the ingestible L-glutamine compound is a peptide containing L-glutamine.

5. The method of claim 1, wherein the ingestible L-glutamine compound is a physiologically acceptable derivative of L-glutamine.

6. The method of claim 1, wherein the L-glutamine compound is administered in the form of an ingestible composition.

7. The method of claim 1, wherein the L-glutamine compound is administered in the form of a tablet, capsule, or concentrate.

8. The method of claim 1, wherein the L-glutamine compound is administered in the form of a liquid drink product.

9. The method of claim 1, wherein the L-glutamine compound is administered in the form of a food

10. The method of claim 1, wherein the subject is a human.

11. The method of claim 1, wherein 10 to 50 g of the ingestible L-glutamine compound is administered to the subject per day.

12. The method of claim 1, wherein 10 to 30 g of the ingestible L-glutamine compound is administered to the subject per day.

13. The method of claim 1, wherein 10 to 20 g of the ingestible L-glutamine compound is administered to the subject per day.

14. The method of claim 1, wherein the ingestible L-glutamine compound is administered prior to the exercise.

15. The method of claim 1, wherein the exercising is conducted at at least 40% VO₂max.

16. The method of claim 1, wherein the exercising is conducted at 40% to 85% VO₂max.

17. The method of claim 1, wherein the exercising is conducted at 40% to 70% VO₂max.

18. The method of claim 1, wherein the exercising is conducted at 50% to 70% VO₂max.

19. The method of claim 1, wherein the exercising is conducted for at least 20 minutes.

20. A method of reducing body weight and/or body fat, comprising:
consuming at least 10 g of an ingestible L-glutamine compound per day; and
physically exercising.

21. The method of claim 20, wherein the ingestible L-glutamine compound is L-glutamine, a peptide containing L-glutamine, or a physiologically acceptable derivative of L-glutamine.

22. The method of claim 20, wherein the ingestible L-glutamine compound is L-glutamine.

23. The method of claim 20, wherein the ingestible L-glutamine compound is a peptide containing L-glutamine.

24. The method of claim 20, wherein the ingestible L-glutamine compound is a physiologically acceptable derivative of L-glutamine.

25. The method of claim 20, wherein the L-glutamine compound is administered in the form of an ingestible composition.

26. The method of claim 20, wherein the L-glutamine compound is administered in the form of a tablet, capsule, or concentrate.

27. The method of claim 20, wherein the L-glutamine compound is administered in the form of a liquid drink product.

28. The method of claim 20, wherein the L-glutamine compound is administered in the form of a food

29. The method of claim 20, wherein the subject is a human.

30. The method of claim 20, wherein 10 to 50 g of the ingestible L-glutamine compound is administered to the subject per day.

31. The method of claim 20, wherein 10 to 30 g of the ingestible L-glutamine compound is administered to the subject per day.

32. The method of claim 20, wherein 10 to 20 g of the ingestible L-glutamine compound is administered to the subject per day.

33. The method of claim 20, wherein the ingestible L-glutamine compound is administered prior to the exercise.

34. The method of claim 20, wherein the exercising is conducted at at least 40% VO₂max.

35. The method of claim 20, wherein the exercising is conducted at 40% to 85% VO₂max.

36. The method of claim 20, wherein the exercising is conducted at 40% to 70% VO₂max.

37. The method of claim 20, wherein the exercising is conducted at 50% to 70% VO₂max.

38. The method of claim 20, wherein the exercising is conducted for at least 20 minutes.

39. A liquid beverage product, comprising at least 5 g of a L-glutamine compound per 473 mL of the beverage product.
